(19) United States
(12) Patent Application Publication
(10) Pub. No.: US 2024/0287570 A1
(43) Pub. Date: Aug. 29, 2024

(54) METHOD FOR ASSESSING DRUG-RESISTANT STENOTROPHOMONAS MALTOPHILIA AND DRUG-RESISTANT STENOTROPHOMONAS MALTOPHILIA ASSESSING SYSTEM

(71) Applicant: China Medical University, Taichung City (TW)

(72) Inventors: Der-Yang Cho, Taichung City (TW); Po-Ren Hsueh, Taichung City (TW); Jiaxin Yu, Taichung City (TW); Chia-Fong Cho, Taichung City (TW)

(73) Assignee: China Medical University, Taichung City (TW)

(21) Appl. No.: 18/174,019

(22) Filed: Feb. 24, 2023

(51) Int. Cl. C12Q 1/04

(52) U.S. Cl. CPC C12Q 1/04

(57) ABSTRACT

A method for assessing drug-resistant Stenotrophomonas maltophilia includes the following steps. A test sample is provided, wherein the test sample includes a Stenotrophomonas maltophilia. A spectrum analysis step is performed, wherein the test sample is detected by a mass spectrometry method so as to obtain a target mass spectrum data. An assessing step for drug-resistant Stenotrophomonas maltophilia is performed, wherein the target mass spectrum data is analyzed so as to assess whether the Stenotrophomonas maltophilia is susceptible or resistant to a ceftazidime antibiotic. When the Stenotrophomonas maltophilia is susceptible to the ceftazidime, the target mass spectrum data includes a first anti-ceftazidime feature mark, and a mass-to-charge ratio of the first anti-ceftazidime feature mark ranges from 5,965 to 5,975 daltons. When the Stenotrophomonas maltophilia is resistant to the ceftazidime, the target mass spectrum data is without a peak at the first anti-ceftazidime feature mark.

### BACKGROUND

### Technical Field

The present disclosure relates to a medical information analysis method and a system thereof. More particularly, the present disclosure relates to a method for assessing drug-resistant Stenotrophomonas maltophilia and a drug-resistant Stenotrophomonas maltophilia assessing system.

### Description of Related Art

Multidrug-resistant gram-negative bacteria (MDR-GNB) have become a serious clinical challenge to the modern society, and Stenotrophomonas maltophilia, a bacillus classified in Xanthomonadaceae, is an MDR-GNB associated with a high mortality rate worldwide.

Stenotrophomonas maltophilia is resistant to a wide range of antibiotics, including β-lactams, carbapenems, fluoroquinolones, and trimethoprim-sulfamethoxazole. In detail, Stenotrophomonas maltophilia can produce hydrolases, including Zinc-containing penicillinase (L1) and Cephalosporinase (L2), to hydrolyze the β-lactams, and the lipopolysaccharide outer member thereof can prevent the Stenotrophomonas maltophilia from being killed by most of the antibiotics. Further, the plasmid in the Stenotrophomonas maltophilia including the drug-resistance genes can be spread between bacteria by conjugation, resulting in higher morbidity or mortality of Stenotrophomonas maltophilia increasing day by day. Furthermore, once the infection of Stenotrophomonas maltophilia is caused in immunocompromised patients, it will be more difficult to treat, and the mortality rate of the patients will increase.

Therefore, how to provide a rapid and accurate method to identify whether Stenotrophomonas maltophilia is susceptible to certain antibiotics or not so as to rapidly and accurately formulate an appropriate treatment strategy, has become the goal of the relevant academic and industry development.

### SUMMARY

According to one aspect of the present disclosure, a method for assessing drug-resistant Stenotrophomonas maltophilia includes the following steps. A test sample is provided, wherein the test sample includes a Stenotrophomonas maltophilia. A spectrum analysis step is performed, wherein the test sample is detected by a mass spectrometry method so as to obtain a target mass spectrum data, and the target mass spectrum data is a mass spectrum data of the Stenotrophomonas maltophilia. An assessing step for drug-resistant Stenotrophomonas maltophilia is performed, wherein the target mass spectrum data is analyzed so as to assess whether the Stenotrophomonas maltophilia is susceptible or resistant to a ceftazidime. When the Stenotrophomonas maltophilia is susceptible to the ceftazidime, the target mass spectrum data includes a first anti-ceftazidime feature mark, and a mass-to-charge ratio of the first anti-ceftazidime feature mark ranges from 5,965 to 5,975 daltons. When the Stenotrophomonas maltophilia is resistant to the ceftazidime, the target mass spectrum data is without a peak at the first anti-ceftazidime feature mark.

According to another aspect of the present disclosure, a drug-resistant Stenotrophomonas maltophilia assessing system includes a non-transitory machine readable medium and a processor. The non-transitory machine readable medium is for storing a target mass spectrum data, wherein the target mass spectrum data is obtained by detecting a test sample with a mass spectrometry method, and the test sample includes a Stenotrophomonas maltophilia. The processor is signally connected to the non-transitory machine readable medium, wherein the processor includes an analyzing module. The analyzing module is for assessing whether the Stenotrophomonas maltophilia is susceptible or resistant to a ceftazidime based on a feature set, wherein the feature set includes a first anti-ceftazidime feature mark, and a mass-to-charge ratio of the first anti-ceftazidime feature mark ranges from 5,965 to 5,975 daltons. When the Stenotrophomonas maltophilia is susceptible to the ceftazidime, the target mass spectrum data includes the first anti-ceftazidime feature mark. When the Stenotrophomonas maltophilia is resistant to the ceftazidime, the target mass spectrum data is without a peak at the first anti-ceftazidime feature mark.

### BRIEF DESCRIPTION OF THE DRAWINGS

The patent or application file contains at least one drawing executed in color. Copies of this patent or patent application publication with color drawing(s) will be provided by Office upon request and payment of the necessary fee. The present disclosure can be more fully understood by reading the following detailed description of the embodiment, with reference made to the accompanying drawings as follows:

FIG. 1 is a flow chart of a method for assessing drug-resistant Stenotrophomonas maltophilia according to one embodiment of the present disclosure.

FIG. 2 is a block diagram of a drug-resistant Stenotrophomonas maltophilia assessing system according to another embodiment of the present disclosure.

FIG. 3 is a flow chart of a method for assessing drug-resistant Stenotrophomonas maltophilia of Example 1.

FIG. 4 shows a summary graph of the results of the SHAP method when assessing the susceptibility of the Stenotrophomonas maltophilia.

FIG. 5 shows one mass spectrometry data of different types of Stenotrophomonas maltophilia.

FIG. 6 shows another mass spectrometry data of different types of Stenotrophomonas maltophilia.

FIG. 7 shows further another mass spectrometry data of different types of Stenotrophomonas maltophilia.

FIG. 8 is a pseudogel plot of all samples used to assess the susceptibility of the Stenotrophomonas maltophilia to the ceftazidime.

### DETAILED DESCRIPTION

The various embodiments of the invention are discussed in more detail below. However, the embodiments may be specific to various applications of the inventive concept and may be practiced in a variety of specific contexts. The particular embodiments are for illustrative purposes only and are not limited to the scope of the disclosure.

[Method for Assessing Drug-Resistant Stenotrophomonas maltophilia of the Present Disclosure]

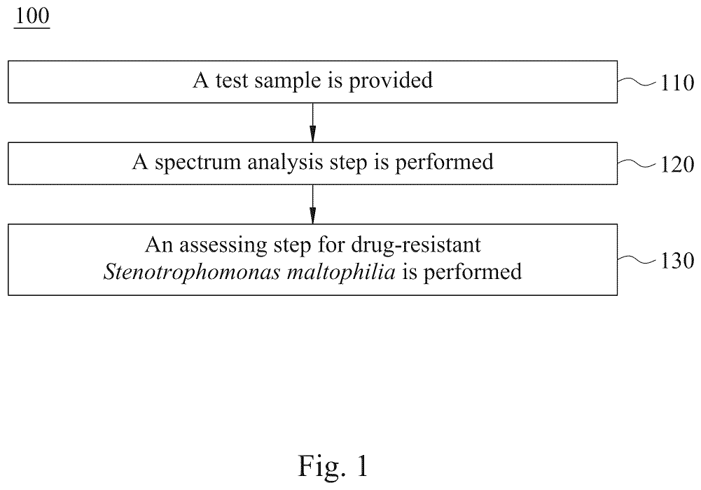

Reference is made to FIG. 1, which is a flow chart of a method 100 for assessing drug-resistant Stenotrophomonas maltophilia according to one embodiment of the present disclosure. The method 100 for assessing drug-resistant Stenotrophomonas maltophilia includes Step 110, Step 120 and Step 130.

In Step 110, a test sample is provided, wherein the test sample includes a Stenotrophomonas maltophilia. Stenotrophomonas maltophilia is a kind of Gram-negative bacteria belonging to the genus Stenotrophomonas of Xanthomonadaceae. In detail, Stenotrophomonas maltophilia can adhere strongly and form biofilm on the mucosal surfaces of the upper respiratory tract and large bronchi, and thus Stenotrophomonas maltophilia usually emerges as an opportunistic nosocomial pathogen. Further, Stenotrophomonas maltophilia also frequently colonizes humid surfaces such as the tubes used in mechanical ventilation and indwelling urinary catheters. Furthermore, due to the anti-drug characteristics of Stenotrophomonas maltophilia, the mortality rate of bacteremia caused by Stenotrophomonas maltophilia is about 30%-70%, especially if it is not properly treated the patient with suitable antibiotics. Accordingly, the Stenotrophomonas maltophilia is used as the target microorganism in the method 100 for assessing drug-resistant Stenotrophomonas maltophilia of the present disclosure so as to respond to the increasing number of the related clinical cases.

In Step 120, a spectrum analysis step is performed, wherein the test sample is detected by a mass spectrometry method so as to obtain a target mass spectrum data, and the target mass spectrum data is a mass spectrum data of the Stenotrophomonas maltophilia. In detail, the mass spectrometry method used in the present disclosure is Matrix Assisted Laser Desorption Ionization Time-of-Flight mass spectrometry (“MALDI-TOF” hereafter). In the MALDI-TOF, the liquid or solid sample is mixed with the detection reagent (substrate), and then the laser is applied to excite the sample to form gas-phase ions. Then, the mass-to-charge ratio of each of the gas-phase ions is detected by a mass spectrometer and then converted into a mass spectrum data. In the clinical application, the mass spectrum data of the sample can be compared with the mass spectrometry database of the microorganisms established by the same mass spectrometry method, so the strains of the microorganisms can be identified based on the principle that the mass spectrum data of the same microorganism is identical. Accordingly, by using the MALDI-TOF method to detect the test sample to obtain the mass spectrum data of the Stenotrophomonas maltophilia, the method 100 for assessing drug-resistant Stenotrophomonas maltophilia of the present disclosure can be close to the current clinical process used to identify microorganisms, and it has not only a high market acceptance but also a high assessment accuracy in the related application.

In Step 130, an assessing step for drug-resistant Stenotrophomonas maltophilia is performed, wherein the target mass spectrum data is analyzed so as to assess whether the Stenotrophomonas maltophilia is susceptible or resistant to a ceftazidime. In detail, the ceftazidime is an injected broad-spectrum third-generation cephalosporin beta-lactam antibiotic used to treat or prevent a variety of bacterial infections, including pneumonia, gynecological infections, bone and joint infections, and septicemia, among others. In the mechanism thereof, the ceftazidime will bind to and inactivate the penicillin-binding proteins (PBP) located on the inner membrane of the bacterial cell wall. The inactivation of PBPs interferes with the cross-linkage of peptidoglycan chains necessary for bacterial cell wall strength and rigidity. This results in the weakening of the bacterial cell wall and causes cell lysis.

Thus, when the Stenotrophomonas maltophilia is susceptible to the ceftazidime, the target mass spectrum data includes a first anti-ceftazidime feature mark, and a mass-to-charge ratio of the first anti-ceftazidime feature mark ranges from 5,965 to 5,975 daltons. In other words, when the ceftazidime has the inhibitory effect on the Stenotrophomonas maltophilia in the test sample, the target mass spectrum data of the test sample will have a peak at the mass-to-charge ratio of 5,965 to 5,975 daltons, and the Stenotrophomonas maltophilia in the test sample can be assessed to be susceptible to the ceftazidime. On the contrary, when the Stenotrophomonas maltophilia is resistant to the ceftazidime, the target mass spectrum data is without a peak at the first anti-ceftazidime feature mark. In other words, when the ceftazidime has no inhibitory effect on the Stenotrophomonas maltophilia in the test sample, the target mass spectrum data of the test sample does not include the first anti-ceftazidime feature mark ranges from 5,965 to 5,975 daltons, and the Stenotrophomonas maltophilia in the test sample can be assessed to be resistant to the ceftazidime. Furthermore, the mass-to-charge ratio of the first anti-ceftazidime feature mark can be 5,970 daltons.

Therefore, the method 100 for assessing drug-resistant Stenotrophomonas maltophilia of the present disclosure can be effectively used to assess whether the Stenotrophomonas maltophilia is susceptible or resistant to the ceftazidime based on the mass spectrometry data thereof, so that a drug susceptibility assessing result of the Stenotrophomonas maltophilia will be output accordingly, the medical strategy in clinical can be timely obtained, and the method 100 for assessing drug-resistant Stenotrophomonas maltophilia has the potential application in relevant markets.

Further, when the Stenotrophomonas maltophilia is resistant to the ceftazidime, the target mass spectrum data can include a second anti-ceftazidime feature mark, a mass-to-charge ratio of the second anti-ceftazidime feature mark ranges from 4,950 to 4,960 daltons, and the target mass spectrum data of the Stenotrophomonas maltophilia of the test sample has a peak at the mass-to-charge ratio of 4,950 to 4,960 daltons. Furthermore, the mass-to-charge ratio of the second anti-ceftazidime feature mark can be 4,955 daltons.

Furthermore, when the Stenotrophomonas maltophilia is resistant to the ceftazidime, the target mass spectrum data can include a third anti-ceftazidime feature mark, a mass-to-charge ratio of the third anti-ceftazidime feature mark ranges from 9,585 to 9,595 daltons, and the target mass spectrum data of the Stenotrophomonas maltophilia of the test sample has a peak at the mass-to-charge ratio of 9,585 to 9,595 daltons. Furthermore, the mass-to-charge ratio of the third anti-ceftazidime feature mark can be 9,590 daltons.

[Drug-Resistant Stenotrophomonas maltophilia Assessing System of the Present Disclosure]

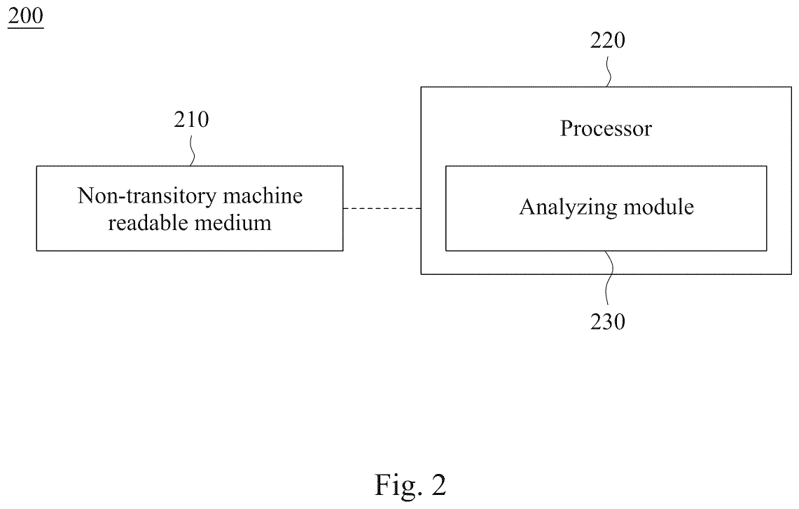

Reference is made to FIG. 2, which is a block diagram of a drug-resistant Stenotrophomonas maltophilia assessing system 200 according to another embodiment of the present disclosure. The drug-resistant Stenotrophomonas maltophilia assessing system 200 includes a non-transitory machine readable medium 210 and a processor 220.

The non-transitory machine readable medium 210 is for storing a target mass spectrum data, wherein the target mass spectrum data is obtained by detecting a test sample with a mass spectrometry method, and the test sample includes a Stenotrophomonas maltophilia.

The processor 220 is signally connected to the non-transitory machine readable medium 210, and the processor 220 includes an analyzing module 230. The analyzing module 230 is for assessing whether the Stenotrophomonas maltophilia is susceptible or resistant to a ceftazidime based on a feature set, wherein the feature set includes a first anti-ceftazidime feature mark, and a mass-to-charge ratio of the first anti-ceftazidime feature mark ranges from 5,965 to 5,975 daltons. When the Stenotrophomonas maltophilia is susceptible to the ceftazidime, the target mass spectrum data includes the first anti-ceftazidime feature mark. When the Stenotrophomonas maltophilia is resistant to the ceftazidime, the target mass spectrum data is without a peak at the first anti-ceftazidime feature mark. Furthermore, the mass-to-charge ratio of the first anti-ceftazidime feature mark can be 5,970 daltons.

Therefore, drug-resistant Stenotrophomonas maltophilia assessing system 200 of the present disclosure can effectively assess whether the Stenotrophomonas maltophilia is susceptible or resistant to the ceftazidime based on that whether the mass spectrum data of the Stenotrophomonas maltophilia includes the first anti-ceftazidime feature mark or not. Accordingly, the time required for the conventional microbial culture, the identification and the antibiotic susceptibility testing can be effectively reduced, and it is favorable for formulating the medical strategy for the drug-resistant Stenotrophomonas maltophilia, and the present disclosure has the potential application in clinical.

Further, the feature set can further include a second anti-ceftazidime feature mark, and when the Stenotrophomonas maltophilia is resistant to the ceftazidime, the target mass spectrum data includes the second anti-ceftazidime feature mark, and a mass-to-charge ratio of the second anti-ceftazidime feature mark ranges from 4,950 to 4,960 daltons. Furthermore, the mass-to-charge ratio of the second anti-ceftazidime feature mark can be 4,955 daltons.

Furthermore, the feature set can further include a third anti-ceftazidime feature mark, and when the Stenotrophomonas maltophilia is resistant to the ceftazidime, the target mass spectrum data includes the third anti-ceftazidime feature mark, and a mass-to-charge ratio of the third anti-ceftazidime feature mark ranges from 9,585 to 9,595 daltons. Furthermore, the mass-to-charge ratio of the third anti-ceftazidime feature mark can be 9,590 daltons.

The method for assessing drug-resistant Stenotrophomonas maltophilia and the drug-resistant Stenotrophomonas maltophilia assessing system of the present disclosure will be further exemplified by performing the following specific embodiments. However, the present disclosure should not be limited to these practical details thereof, that is, in some embodiments, these practical details are used to describe how to implement the materials and methods of the present disclosure and are not necessary.

### Example 1

In order to assess the efficacy of each of the anti-ceftazidime feature marks of the present disclosure for assessing whether the Stenotrophomonas maltophilia is susceptible or resistant to the ceftazidime, the following specific testing examples will be further exemplified.

### I. Database

The database of Example 1 includes 817 of the reference mass spectrum data, wherein the reference mass spectrum data are obtained after the samples infected by the Stenotrophomonas maltophilia are processed by a conventional sample processing method in the clinical laboratory, and the reference mass spectrum data in the database are MALDI-TOF data so as to be close to the current clinical process used to identify microorganisms. The Stenotrophomonas maltophilia used in Example 1 includes ceftazidime-susceptible Stenotrophomonas maltophilia (227 of 817) and ceftazidime-resistant Stenotrophomonas maltophilia (590 of 817). In detail, the susceptibilities of ceftazidime of the Stenotrophomonas maltophilia is determined using the broth microdilution method and is interpreted according to the Clinical and Laboratory Standard Institute (CLSI) guidelines to determine the minimum inhibitory concentrations (MICs) of antibiotics against the Stenotrophomonas maltophilia isolates. Further, compared to the broth microdilution method, the conventional automated antibiotic susceptibility test (AST) system for the Stenotrophomonas maltophilia to the ceftazidime in clinical, including Phoenix susceptibility system (Becton-Dickinson Diagnostic Systems, Sparks, MD, USA) and Vitek 2 (bioMerieux, Durham NC), can greatly reduce time and manpower. Thus, when the Stenotrophomonas maltophilia is not susceptible to the ceftazidime according to the results of any of the aforementioned testing methods, it is assessed to have ceftazidime resistance.

### II. Analysis the Contributions of Different Anti-Ceftazidime Feature Marks

In Example 1, the reference mass spectrum data of the database will be analyzed by an antibiotic resistance assessing classifier first so as to find the characteristic feature marks which can be used as the feature marks of the Stenotrophomonas maltophilia being resistant to the ceftazidime.

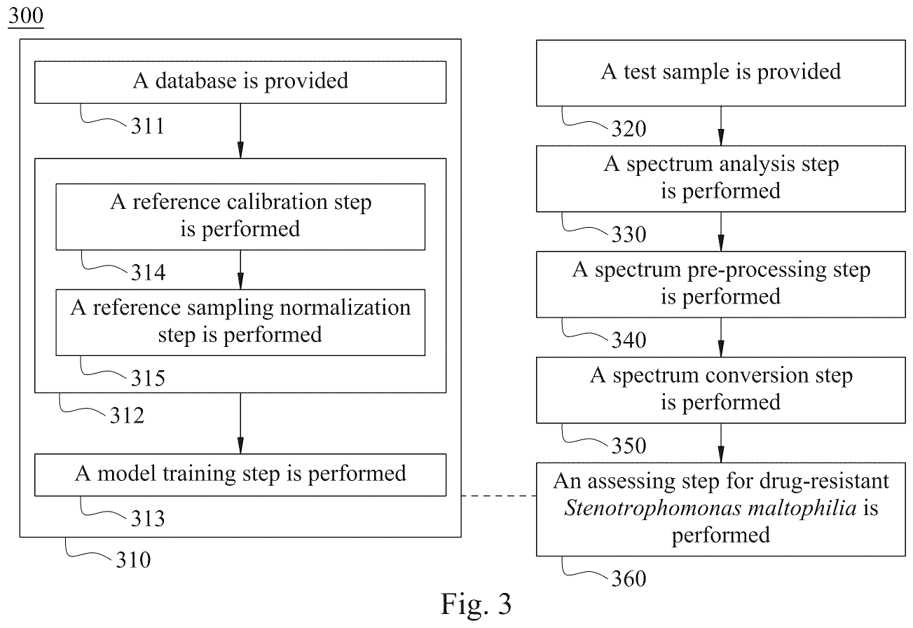

Reference is made to FIG. 3, which is a flow chart of a method 300 for assessing drug-resistant Stenotrophomonas maltophilia of Example 1. The method 300 for assessing drug-resistant Stenotrophomonas maltophilia includes Step 310, Step 320, Step 330, Step 340, Step 350 and Step 360, wherein Step 320, Step 330 and Step 360 are the same to Step 110, Step 120 and Step 130 of FIG. 1, so that the details thereof will not be described herein again.

In Step 310, a model establishing step is performed, wherein the model establishing step is for establishing the antibiotic resistance assessing classifier, and the antibiotic resistance assessing classifier is for extracting the characteristic feature marks used in the present disclosure and analyzing the importance of different characteristic feature marks for drug susceptibility assessment of the Stenotrophomonas maltophilia to the ceftazidime. The model establishing step includes Step 311, Step 312 and Step 313.

In Step 311, a database is provided. In detail, the database includes the plurality reference mass spectrum data being MALDI-TOF mass spectrum data.

In Step 312, a reference spectrum pre-processing step is performed, wherein all of the reference mass spectrum data are pre-processed so as to obtain a plurality of normalized reference mass spectrum data. In detail, Python (version 3.7) is served as the spectrum pre-processing module of the present disclosure, and the reference spectrum pre-processing step can include Step 314 and Step 315.

In Step 314, a reference calibration step is performed, wherein a background noise of each of the reference mass spectrum data is removed. In detail, before the reference calibration step is performed, each of the reference mass spectrum data will be examined initially so as to exclude the mass spectrum data including blank portions or error formats. Then, each of the reference mass spectrum data will be smoothed so as to remove the background noise thereof.

In Step 315, a reference sampling normalization step is performed, wherein a temporal resolution value of each of the reference mass spectrum data is adjusted so as to obtain the plurality of normalized reference mass spectrum data. In detail, in the reference sampling normalization step, the raw signal resolution and the sampling frequency of all of the reference mass spectrum data will be respectively checked so as to check whether there is an inconsistency or not. If so, all the reference mass spectrum data will be resampled so as to make the temporal resolution values thereof consistent. Further, the baseline of each of the reference mass spectrum data will be corrected by the top-hat method and then normalized according to the following Formula (I), so that the signal resolution of all the reference mass spectrum data can be consistent. Formula (I) is shown below:

$$z = ( x - μ ) / σ ; \qquad Formula ( I )$$

wherein “z” represents z-score, “x” represents the mass-to-charge ratio intensity of each point of the reference mass spectrum data, “u” represents the average signal intensity of the reference mass spectrum data, and “o” represents the standard deviation of intensity of the reference mass spectrum data. After normalizing the intensity of the reference mass spectrum data, the mass-to-charge ratio data with negative z-scores represents that the subtle signals or the noise are existed, and the said reference mass spectrum data will be further removed. Thus, the normalized reference mass spectrum data without the background noise and the sampling frequency and the intensity thereof being normalized can be obtained.

Further, in the reference sampling normalization step, the reference mass spectrum data is further processed by a mass-to-charge ratio conversing method. In the spectrum conversion step, a data interval value of the reference mass spectrum data can be further adjusted by a binning method so as to prevent the peaks of the reference mass spectrum data from shifting due to the effects of the isotopes in the mass spectrometry method. Preferably, the data interval value of the reference mass spectrum data can range from 1 to 10 daltons, and more preferably, the data interval value of the reference mass spectrum data can be 5 daltons or 10 daltons.

In Step 313, a model training step is performed, wherein the plurality of the normalized reference mass spectrum data are trained to achieve a convergence by an algorithm classifier so as to obtain the antibiotic resistance assessing classifier. Preferably, the algorithm classifier can be LightGBM (Light Gradient Boosting Machine) algorithm classifier, CatBoost algorithm classifier, XGBoost (Extreme Gradient Boosting) algorithm classifier, Gradient Boosting algorithm classifier or other algorithm classifiers based on the decision tree algorithm, but the present disclosure is not limited thereto. More preferably, the algorithm classifier can be LightGBM algorithm classifier, so that the robustness and the generality of the antibiotic resistance assessing classifier can be maintained, and the antibiotic resistance assessing classifier will be used to extract the feature marks and then analyze the importance of different feature marks of the target mass spectrum data to the assessment of the drug-resistant Stenotrophomonas maltophilia.

Then, the feature marks used in the present disclosure can be obtained by the analysis of the antibiotic resistance assessing classifier, and the feature marks obtained therefrom will be used in the method for assessing drug-resistant Stenotrophomonas maltophilia and the drug-resistant Stenotrophomonas maltophilia assessing system of the present disclosure as the base to assess whether the Stenotrophomonas maltophilia is susceptible or resistant to the ceftazidime.

Moreover, the target mass spectrum data of the present disclosure will also be pre-processed before used to assess the drug-resistant Stenotrophomonas maltophilia, and the pre-processing details are shown in Step 340 and Step 350.

In Step 340, a spectrum pre-processing step is performed, wherein the target mass spectrum data is pre-processed so as to obtain a processed target mass spectrum data, and the spectrum pre-processing step can include a calibration step and a sampling normalization step. In the calibration step, the signals of the target mass spectrum data will be smoothed so as to remove the background noise thereof. In the sampling normalization step, a temporal resolution value of the target mass spectrum data is adjusted so as to obtain the processed target mass spectrum data.

In greatly detail, in the sampling normalization step, the raw signal resolution and the sampling frequency of the target mass spectrum data processed by the calibration step will be checked whether there is an inconsistency or not. If so, the target mass spectrum data will be resampled in the sampling normalization step so as to make the temporal resolution values thereof consistent. Then, a baseline correction will be performed by the top-hat method and then normalized according to the aforementioned Formula (I) so as to make the signal resolution consistent. The details of Formula (I) are shown in the aforementioned paragraph and will not be described herein again.

In Step 350, a spectrum conversion step is performed, wherein the processed target mass spectrum data is processed by a mass-to-charge ratio conversing method so as to obtain a normalized target mass spectrum data. Further, in the spectrum conversion step, a data interval value of the normalized target mass spectrum data can be further adjusted by a binning method so as to prevent the peaks of the normalized target mass spectrum data from shifting due to the effects of the isotopes in the mass spectrometry method. Preferably, the data interval value of the normalized target mass spectrum data can range from 1 to 10 daltons, and more preferably, the data interval value of the converted mass spectrum data can be 5 daltons or 10 daltons.

In Example 1, a total of 3 feature marks having the mass-to-charge ratios with different ranges are used to assess the antibiotic susceptibility of the drug-resistant Stenotrophomonas maltophilia to the ceftazidime. The details of the 3 feature marks are shown in Table 1, and the SHapely Additive explanations method (“SHAP method” hereafter) is applied for illustrating the contributions of the 3 feature marks used to assess the antibiotic susceptibility of the drug-resistant Stenotrophomonas maltophilia to the ceftazidime.

TABLE 1

| | | Mass-to-charge ratio |
|---|---|---|
| Feature mark | Feature | (Daltons) |
| 5970 | 772, 773, 774 | 5,965 to 5,975 |
| 4955 | 571 | 4,950 to 4,960 |
| 9590 | 1498 | 9,585 to 9,595 |

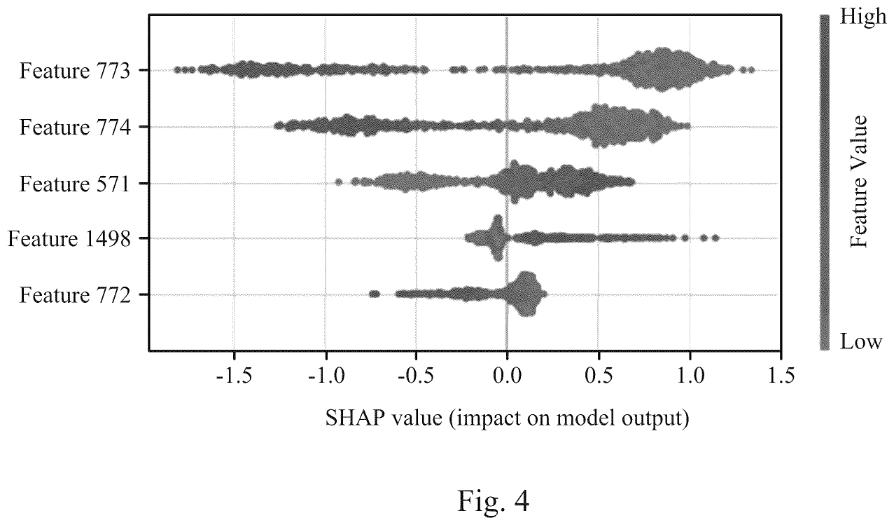

Reference is made to FIG. 4, which shows a summary graph of the results of the SHAP method when assessing the susceptibility of the Stenotrophomonas maltophilia. In FIG. 4, the X-axis represents the SHAP values, which are used to judge the influence of a feature on the output of a model, and the Y-axis represents all the features used to assess the susceptibility of the Stenotrophomonas maltophilia to the ceftazidime, wherein Features 772, 773 and 774 locate in a range of m/z 5,965 to 5,975 (that is, Feature mark 5970), Feature 571 locates in a range of m/z 4,950 to 4,960 (that is, Feature mark 4955), and Feature 1498 locates in a range of m/z 9,585 to 9,595 (that is, Feature mark 9590).

In FIG. 4, each point is one SHAP value for a prediction of a feature, wherein the red color represents the higher value of the feature, and the blue color represents the lower value of the feature. Further, a positive SHAP value means a positive impact on the prediction, and a negative SHAP value means a negative impact on the prediction. As shown in FIG. 4, the sequence in order from the top to the bottom presents the importance range of the features, and each of the feature marks independently presents the contribution thereof to the assessment of the drug susceptibility and does not affect each other's contribution, wherein Feature 773 is the most important, followed by Feature 774, Feature 571, Feature 1498 and Feature 772.

In detail, take Feature 773 as an example, when a feature value of Feature 773 is higher (the red color in FIG. 4, wherein the feature value represents the peak intensity of Feature 773), the lower the probability of Feature 773 for predicting the resistance of the Stenotrophomonas maltophilia (the SHAP value thereof is negative) to the ceftazidime, and when a feature value of Feature 773 is lower (the blue color in FIG. 4), the higher the probability of Feature 773 for predicting the resistance of the Stenotrophomonas maltophilia to the ceftazidime (the SHAP value thereof is positive). The same trend is also shown in Features 774 and 772. Thus, Feature mark 5970 including Features 773, 774 and 772 is used as the first anti-ceftazidime feature mark of the present disclosure, wherein the first anti-ceftazidime feature mark is the most important feature for assessing the drug susceptibility of the Stenotrophomonas maltophilia, and when the Stenotrophomonas maltophilia is resistant to the ceftazidime, the target mass spectrum data is without a peak at the first anti-ceftazidime feature mark. Further, Feature mark 4955 including Feature 571 and Feature mark 9590 including Feature 1498 are respectively used as the second anti-ceftazidime feature mark and the third anti-ceftazidime feature mark of the present disclosure.

III. The efficacy of the method for assessing drug-resistant Stenotrophomonas maltophilia and the drug-resistant Stenotrophomonas maltophilia assessing system of the present disclosure used to assess the antibiotic susceptibility of the drug-resistant Stenotrophomonas maltophilia to the ceftazidime

After the target mass spectrum data is analyzed by the drug-resistant Stenotrophomonas maltophilia assessing system of the present disclosure according to the method for assessing drug-resistant Stenotrophomonas maltophilia of the present disclosure, the accuracy, the AUROC (area under the receiver operating characteristic curve), the recall values, the precision, the F1 score and MCC (Matthews correlation coefficient) under different data interval values are shown in Table 2.

TABLE 2

| | | | | Preci- | F1 | |
|---|---|---|---|---|---|---|
| | Accuracy | AUROC | Recall | sion | score | MCC |
| 5 Daltons | 0.8659 | 0.9231 | 0.9605 | 0.8655 | 0.9106 | 0.6594 |
| 10 Daltons | 0.8927 | 0.9423 | 0.9391 | 0.9143 | 0.9265 | 0.7285 |

Thus, it is shown that the method for assessing drug-resistant Stenotrophomonas maltophilia and the drug-resistant Stenotrophomonas maltophilia assessing system of the present disclosure can effectively assess whether the Stenotrophomonas maltophilia is susceptible or resistant to the ceftazidime.

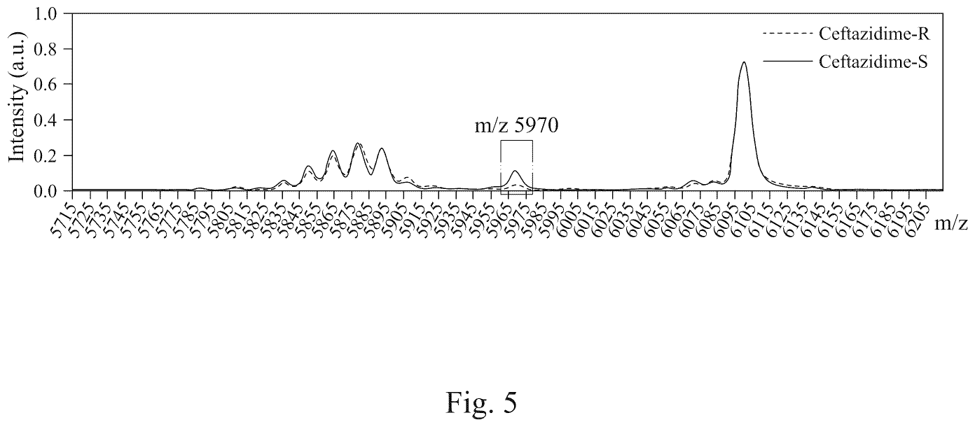

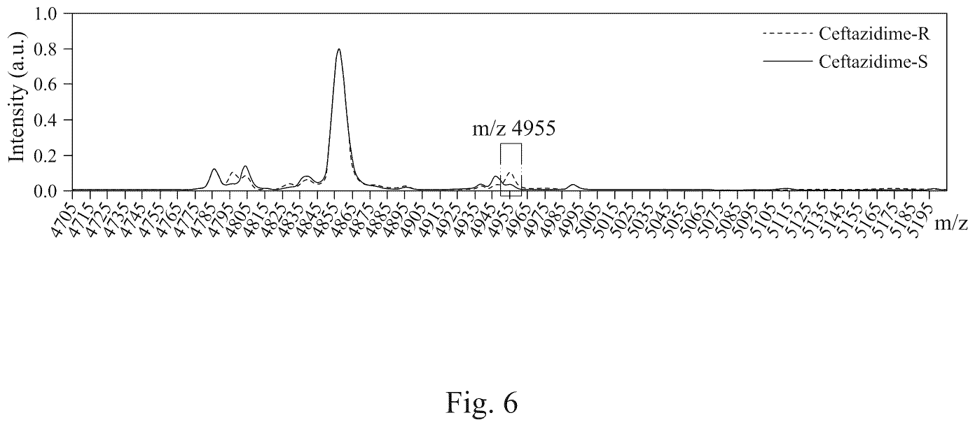

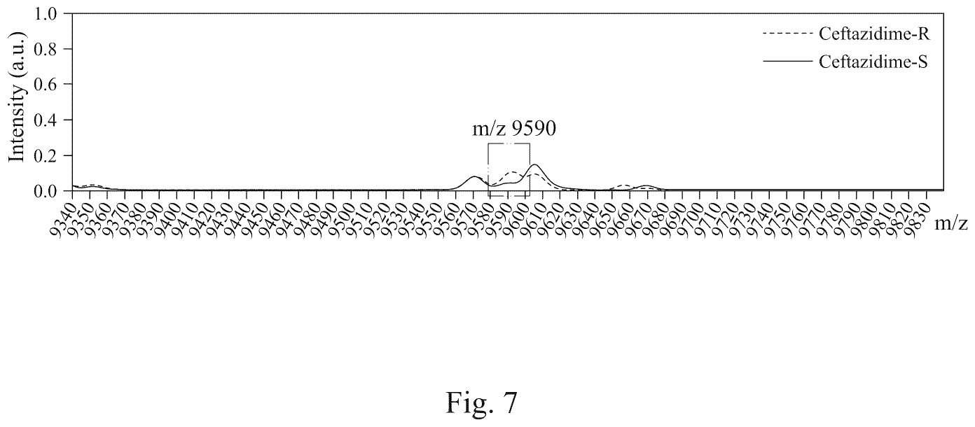

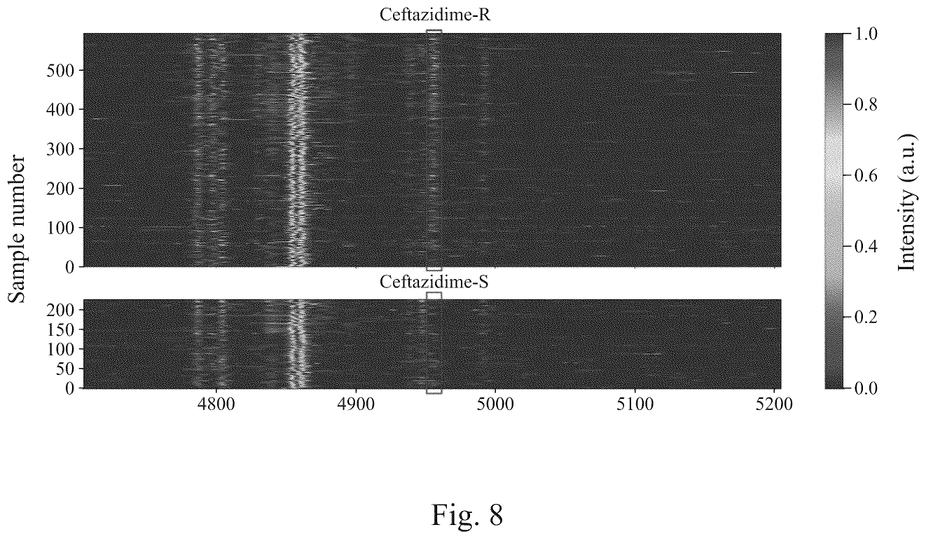

Reference is made to FIG. 5, FIG. 6, FIG. 7 and FIG. 8. FIG. 5 shows one mass spectrometry data of different types of Stenotrophomonas maltophilia, FIG. 6 shows another mass spectrometry data of different types of Stenotrophomonas maltophilia, FIG. 7 shows further another mass spectrometry data of different types of Stenotrophomonas maltophilia, and FIG. 8 is a pseudogel plot of all samples used to assess the susceptibility of the Stenotrophomonas maltophilia to the ceftazidime.

In FIG. 5 to FIG. 7, the x-axis represents the mass-to-charge ratio value of the mass spectrum data, and the y-axis represents the intensity of the peak. As shown in FIG. 5, the mass spectrum data of ceftazidime-susceptible Stenotrophomonas maltophilia (Ceftazidime-S in FIGS. 5 to 8) has a peak at the mass-to-charge ratio of 5,965 to 5,975 daltons (that is, “m/z 5970”), but the mass spectrum data of ceftazidime-resistant Stenotrophomonas maltophilia (Ceftazidime-R in FIGS. 5 to 8) is without a peak at the mass-to-charge ratio of 5,965 to 5,975 daltons. As shown in FIG. 6, compared with the mass spectrum data of ceftazidime-susceptible Stenotrophomonas maltophilia, the mass spectrum data of ceftazidime-resistant Stenotrophomonas maltophilia has a peak at the mass-to-charge ratio of 4,950 to 4,960 daltons (that is, “m/z 4955”). Further, as shown in FIG. 7, compared with the mass spectrum data of ceftazidime-susceptible Stenotrophomonas maltophilia, the mass spectrum data of ceftazidime-resistant Stenotrophomonas maltophilia has a peak at the mass-to-charge ratio of 9,585 to 9,595 daltons (that is, “m/z 9590”).

Further, as shown in FIG. 8, the x-axis represents the mass-to-charge ratio value of the mass spectrum data, the y-axis represents the sample number, and compared with the samples of ceftazidime-susceptible Stenotrophomonas maltophilia, the samples of ceftazidime-resistant Stenotrophomonas maltophilia have a signal performance in the mass-to-charge ratio of 4,950 to 4,960 daltons (that is, the second anti-ceftazidime feature mark marked in red box).

Therefore, the method for assessing drug-resistant Stenotrophomonas maltophilia and the drug-resistant Stenotrophomonas maltophilia assessing system of the present disclosure can efficiently identify that the samples include the first anti-ceftazidime feature mark with the mass-to-charge ratio of 5,965 to 5,975 daltons, the second anti-ceftazidime feature mark with the mass-to-charge ratio of 4,950 to 4,960 daltons, and the third anti-ceftazidime feature mark with the mass-to-charge ratio of 9,585 to 9,595 daltons or not based on the raw data of the mass spectrometry data thereof during the assessing process. Accordingly, the present disclosure can be used to assess whether the Stenotrophomonas maltophilia is susceptible or resistant to the ceftazidime, the accuracy thereof can be greatly enhanced, and the present disclosure has application potential in related markets.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

1. A method for assessing drug-resistant Stenotrophomonas maltophilia, comprising:
providing a test sample, wherein the test sample comprises a Stenotrophomonas maltophilia;
performing a spectrum analysis step, wherein the test sample is detected by a mass spectrometry method so as to obtain a target mass spectrum data, and the target mass spectrum data is a mass spectrum data of the Stenotrophomonas maltophilia; and
performing an assessing step for drug-resistant Stenotrophomonas maltophilia, wherein the target mass spectrum data is analyzed so as to assess whether the Stenotrophomonas maltophilia is susceptible or resistant to a ceftazidime;
wherein when the Stenotrophomonas maltophilia is susceptible to the ceftazidime, the target mass spectrum data comprises a first anti-ceftazidime feature mark, and a mass-to-charge ratio of the first anti-ceftazidime feature mark ranges from 5,965 to 5,975 daltons;
wherein when the Stenotrophomonas maltophilia is resistant to the ceftazidime, the target mass spectrum data is without a peak at the first anti-ceftazidime feature mark.

2. The method of claim 1, wherein the mass-to-charge ratio of the first anti-ceftazidime feature mark is 5,970 daltons.

3. The method of claim 1, wherein when the Stenotrophomonas maltophilia is resistant to the ceftazidime, the target mass spectrum data comprises:
a second anti-ceftazidime feature mark, wherein a mass-to-charge ratio of the second anti-ceftazidime feature mark ranges from 4,950 to 4,960 daltons.

4. The method of claim 3, wherein the mass-to-charge ratio of the second anti-ceftazidime feature mark is 4,955 daltons.

5. The method of claim 1, wherein when the Stenotrophomonas maltophilia is resistant to the ceftazidime, the target mass spectrum data comprises:
a third anti-ceftazidime feature mark, wherein a mass-to-charge ratio of the third anti-ceftazidime feature mark ranges from 9,585 to 9,595 daltons.

6. The method of claim 5, wherein the mass-to-charge ratio of the third anti-ceftazidime feature mark is 9,590 daltons.

7. A drug-resistant Stenotrophomonas maltophilia assessing system, comprising:
a non-transitory machine readable medium for storing a target mass spectrum data, wherein the target mass spectrum data is obtained by detecting a test sample with a mass spectrometry method, and the test sample comprises a Stenotrophomonas maltophilia; and
a processor signally connected to the non-transitory machine readable medium, wherein the processor comprises:
an analyzing module for assessing whether the Stenotrophomonas maltophilia is susceptible or resistant to a ceftazidime based on a feature set, wherein the feature set comprises a first anti-ceftazidime feature mark, and a mass-to-charge ratio of the first anti-ceftazidime feature mark ranges from 5,965 to 5,975 daltons;
wherein when the Stenotrophomonas maltophilia is susceptible to the ceftazidime, the target mass spectrum data comprises the first anti-ceftazidime feature mark;
wherein when the Stenotrophomonas maltophilia is resistant to the ceftazidime, the target mass spectrum data is without a peak at the first anti-ceftazidime feature mark.

8. The drug-resistant Stenotrophomonas maltophilia assessing system of claim 7, wherein the mass-to-charge ratio of the first anti-ceftazidime feature mark is 5,970 daltons.

9. The drug-resistant Stenotrophomonas maltophilia assessing system of claim 7, wherein the feature set further comprises a second anti-ceftazidime feature mark, and when the Stenotrophomonas maltophilia is resistant to the ceftazidime, the target mass spectrum data comprises the second anti-ceftazidime feature mark, and a mass-to-charge ratio of the second anti-ceftazidime feature mark ranges from 4,950 to 4,960 daltons.

10. The drug-resistant Stenotrophomonas maltophilia assessing system of claim 9, wherein the mass-to-charge ratio of the second anti-ceftazidime feature mark is 4,955 daltons.

11. The drug-resistant Stenotrophomonas maltophilia assessing system of claim 7, wherein the feature set further comprises a third anti-ceftazidime feature mark, and when the Stenotrophomonas maltophilia is resistant to the ceftazidime, the target mass spectrum data comprises the third anti-ceftazidime feature mark, and a mass-to-charge ratio of the third anti-ceftazidime feature mark ranges from 9,585 to 9,595 daltons.

12. The drug-resistant Stenotrophomonas maltophilia assessing system of claim 11, wherein the mass-to-charge ratio of the third anti-ceftazidime feature mark is 9,590 daltons.
